(19) United States
(12) Patent Application Publication
(10) Pub. No.: US 2017/0043046 A1
(43) Pub. Date: Feb. 16, 2017

(54) Air Freshener Assembly

(71) Applicant: Sean Martin, Pickering (CA)

(72) Inventor: Sean Martin, Pickering (CA)

(21) Appl. No.: 14/821,866

(22) Filed: Aug. 10, 2015

(51) Int. Cl. A61L 9/12; B01D 46/00

(52) U.S. Cl. CPC A61L 9/12; B01D 46/0038

(57) ABSTRACT

An air freshener assembly for releasing a chemical scent into an area includes a vent that may be positioned within an air duct. A filter is removably coupled to the vent.

The filter is infused with a chemical scent such that the filter may release the chemical scent into the air urged by the air duct.

### BACKGROUND OF THE DISCLOSURE

### Field of the Disclosure

The disclosure relates to air freshener devices and more particularly pertains to a new air freshener device for releasing a scent into an area.

### SUMMARY OF THE DISCLOSURE

An embodiment of the disclosure meets the needs presented above by generally comprising a vent that may be positioned within an air duct. A filter is removably coupled to the vent. The filter is infused with a chemical scent such that the filter may release the chemical scent into the air urged by the air duct.

There has thus been outlined, rather broadly, the more important features of the disclosure in order that the detailed description thereof that follows may be better understood, and in order that the present contribution to the art may be better appreciated.

There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF THE DRAWINGS

The disclosure will be better understood and objects other than those set forth above will become apparent when consideration is given to the following detailed description thereof. Such description makes reference to the annexed drawings wherein:

FIG. 1 is a perspective view of an air freshener assembly according to an embodiment of the disclosure.

FIG. 2 is a top perspective view of an embodiment of the disclosure.

FIG. 3 is a front view of an embodiment of the disclosure.

FIG. 4 is a right side view of an embodiment of the disclosure.

FIG. 5 is a top view of an embodiment of the disclosure.

### DESCRIPTION OF THE PREFERRED EMBODIMENT

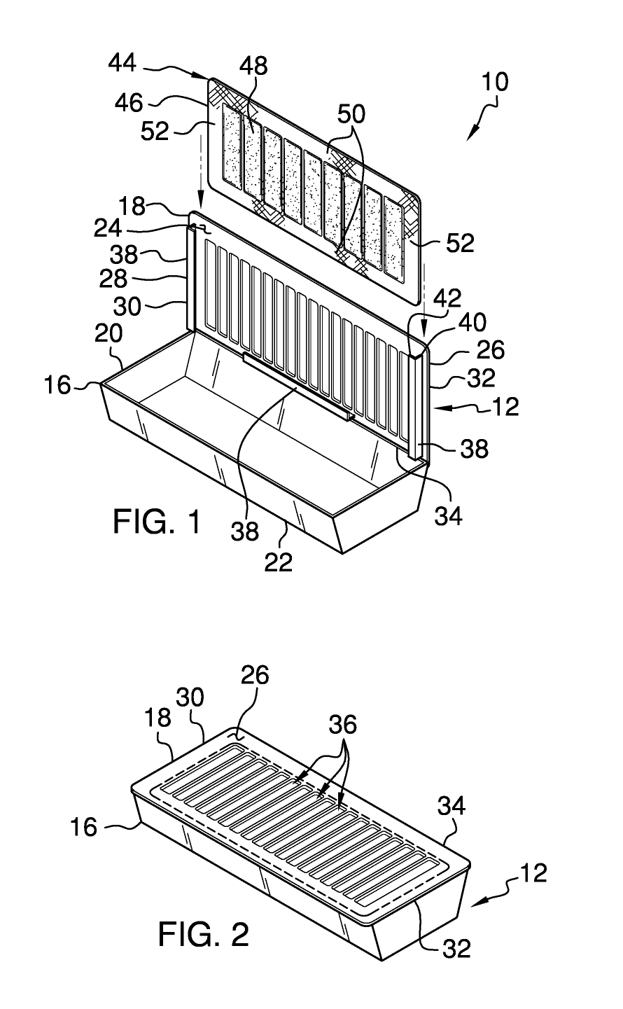

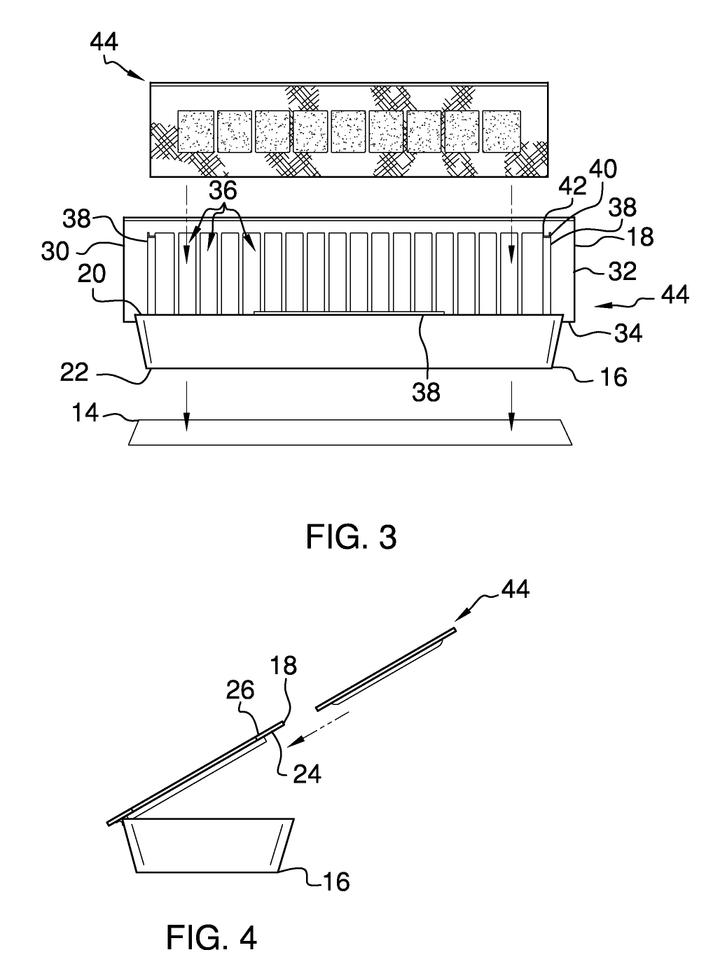

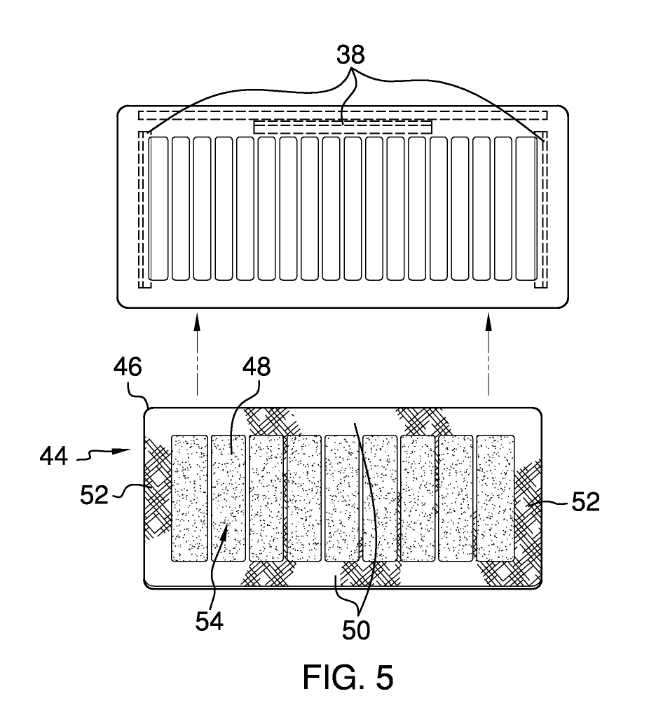

With reference now to the drawings, and in particular to FIGS. 1 through 5 thereof, a new air freshener device embodying the principles and concepts of an embodiment of the disclosure and generally designated by the reference numeral 10 will be described.

As best illustrated in FIGS. 1 through 5, the air freshener assembly 10 generally comprises a vent 12 that may be positioned within an air duct 14. The vent 12 includes a box 16 and a lid 18 that is hingedly coupled to the box 16. The box 16 has a top end 20 and a bottom end 22 and each of the top end 20 and the bottom end 22 are open. The box 16 may be positioned within the air duct 14. The air duct 14 may be a heater vent in a building or the like. The top end 20 has a length and a width that is greater than a length and a width of the bottom end 22 such that the box 16 has a trapezoidal shape. Thus, the box 16 frictionally engages the air duct 14 thereby retaining the box 16 within the air duct 14.

The lid 18 has a bottom surface 24, a top surface 26 and a peripheral edge 28 extending between the top surface 26 and the bottom surface 24. The peripheral edge 28 has a first lateral side 30, a second lateral side 32 and a back side 34. The back side 34 is hingedly coupled to the box 16 such that the lid 18 selectively covers the top end 20. The lid 18 has a plurality of vents 36 extending through the top surface 26 and the bottom surface 24. The vents 36 release air urged by the air duct 14.

The lid 18 includes a plurality of retainers 38 and each of the retainers 38 is coupled to and extends away from the bottom surface 24. Each of the retainers 38 comprises a leg 40 forming an angle with a foot 42. The leg 40 is coupled to the bottom surface 24 such that the foot 42 is spaced from the bottom surface 24. Each of the retainers 38 is aligned with an associated one of the first lateral side 30, the second lateral side 32 and the back side 34.

A filter 44 is provided and the filter 44 is removably coupled to the vent 36. The filter 44 is infused with a chemical scent such that the filter 44 releases the chemical scent into the air urged by the air duct 14. The chemical scent may comprise a floral scent or the like. The filter 44 includes a frame 46 and an element 48. The frame 46 has a pair of lateral arms 50 extending between a pair of longitudinal arms 52 and the lateral arms 50 are spaced from each other to define an opening 54 in the frame 46.

The element 48 is coupled to the frame 46 such that the element 48 completely covers the frame 46. The element 48 is comprised of a fluid permeable material such that the element 48 may allow air to pass through the element 48 while capturing particles in the air. The filter 44 is slid along the bottom surface 24 of the lid 18 such that the frame 46 slides between the foot 42 of each of the retainers 38 and the bottom surface 24. The retainers 38 retain the filter 44 on the lid 18 having the element 48 being aligned with the vents 36.

In use, the box 16 is positioned within the air duct 14. The filter 44 is slid into the retainers 38 and the lid 18 is closed. The element 48 filters air urged by the air duct 14 and the chemical scent is released into the air. The filter 44 is changed when the filter 44 becomes dirty or when the chemical scent becomes depleted.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. An air freshener assembly comprising:
a vent configured to be positioned within an air duct; and
a filter being removably coupled to said vent, said filter being infused with a chemical scent wherein said filter is configured to release said chemical scent into the air urged by the air duct.

2. The assembly according to claim 1, wherein said vent has a box and a lid being hingedly coupled to said box, said box having a top end and a bottom end, each of said top end and said bottom end being open, said box being configured to be positioned within the air duct.

3. The assembly according to claim 1, wherein:
said box has a top end; and
said lid has a bottom surface, a top surface and a peripheral edge extending between said top surface and said bottom surface, said peripheral edge having a first lateral side, a second lateral side and a back side, said back side being hingedly coupled to said box such that said lid selectively covers said top end, said lid having a plurality of vents extending through said top surface and said bottom surface wherein said vents are configured to release air urged by the air duct.

4. The assembly according to claim 3, wherein said lid has a plurality of retainers, each of said retainers being coupled to and extending away from said bottom surface, each of said retainers comprising a leg forming an angle with a foot, said leg being coupled to said bottom surface such that said foot is spaced from said bottom surface, each of said retainers being aligned with an associated one of said first lateral side, said second lateral side and said back side.

5. The assembly according to claim 1, wherein said filter includes a frame and an element, said frame having a pair of lateral arms extending between a pair of longitudinal arms, said lateral arms being spaced from each other to define an opening in said frame.

6. The assembly according to claim 5, wherein said element is coupled to said frame such that said element completely covers said frame, said element being comprised of a fluid permeable material wherein said element is configured to allow air to pass through said element.

7. The assembly according to claim 1, wherein:
said lid having a plurality of retainers, each of said retainers having a foot; and
said filter is slid along said bottom surface of said lid such that said frame slides between said leg of each of said retainers, said retainers retaining said filter on said lid having said element being aligned with said vents.

8. An air freshener assembly comprising:
a vent configured to be positioned within an air duct, said vent having a box and a lid being hingedly coupled to said box, said box having a top end and a bottom end, each of said top end and said bottom end being open, said box being configured to be positioned within the air duct, said lid having a bottom surface, a top surface and a peripheral edge extending between said top surface and said bottom surface, said peripheral edge having a first lateral side, a second lateral side and a back side, said back side being hingedly coupled to said box such that said lid selectively covers said top end, said lid having a plurality of vents extending through said top surface and said bottom surface wherein said vents are configured to release air urged by the air duct, said lid having a plurality of retainers, each of said retainers being coupled to and extending away from said bottom surface, each of said retainers comprising a leg forming an angle with a foot, said leg being coupled to said bottom surface such that said foot is spaced from said bottom surface, each of said retainers being aligned with an associated one of said first lateral side, said second lateral side and said back side; and
a filter being removably coupled to said vent, said filter being infused with a chemical scent wherein said filter is configured to release said chemical scent into the air urged by the air duct, said filter including a frame and an element, said frame having a pair of lateral arms extending between a pair of longitudinal arms, said lateral arms being spaced from each other to define an opening in said frame, said element being coupled to said frame such that said element completely covers said frame, said element being comprised of a fluid permeable material wherein said element is configured to allow air to pass through said element, said filter being slid along said bottom surface of said lid such that said frame slides between said foot of each of said retainers, said retainers retaining said filter on said lid having said element being aligned with said vents.
